(19) United States
(12) Patent Application Publication
(10) Pub. No.: US 2021/0267942 A1
(43) Pub. Date: Sep. 2, 2021

(54) MELATONIN FOR PREVENTING AND TREATING RADIATION VAGINITIS AND PROCTITIS

(71) Applicant: RepoCeuticals ApS, Hørsholm (DK)

(72) Inventor: Lars Otto Uttenthal, Madrid (ES)

(21) Appl. No.: 17/077,406

(22) Filed: Oct. 22, 2020

Related U.S. Application Data
(63) Continuation of application No. 15/553,562, filed on Aug. 24, 2017, now abandoned.

(30) Foreign Application Priority Data
Mar. 6, 2015 (DK) PA201570128

(51) Int. Cl. A61K 31/4045; A61K 31/355; A61K 31/375; A61K 31/385; A61K 31/122; A61K 31/167; A61K 9/00; A61K 9/12; A61K 45/06

(52) U.S. Cl. CPC A61K 31/4045; A61K 31/355; A61K 31/375; A61K 31/385; A61K 31/122; A61K 45/06; A61K 9/0014; A61K 9/0031; A61K 9/0034; A61K 9/122; A61K 31/167

(57) ABSTRACT

Compositions comprising melatonin or derivatives thereof are provided for topical administration to the vaginal and/or rectal epithelium to protect against vaginal and/or rectal radiation injury due to radiotherapy.

### CROSS-REFERENCE TO RELATED APPLICATIONS

This application is a Continuation of and claims the benefit and priority to U.S. patent application Ser. No. 15/553,562, filed on Aug. 24, 2017, which is a U.S. National Phase Application of PCT International Application Number PCT/EP2016/054795, filed on Mar. 7, 2016, designating the United States of America and published in the English language, which is an International Application of and claims the benefit of priority to Danish Patent Application No. PA201570128, filed on Mar. 6, 2015. The disclosures of the above-referenced applications are hereby expressly incorporated by reference in their entireties.

### FIELD OF INVENTION

The present invention provides compositions comprising melatonin or a derivative thereof as the essential ingredient for preventing and treating vaginal or rectal injury due to ionizing radiation by the topical application of the composition to the vaginal or rectal mucosa. As such, it is particularly relevant to the fields of gynecological, colo-rectal and prostatic oncology and radiotherapy, as well as surgery when this is part of a combined treatment with radiotherapy.

### BACKGROUND OF THE INVENTION

### Vaginal Radiation Injury (Radiation Vaginitis)

Radiation injury to the vagina commences immediately on exposure to ionizing radiation. Free radicals are produced in the cells which exceed the cells' intrinsic scavenging capabilities, affecting in particular cells of higher mitotic turnover rate, such as the basal cells of the vaginal epithelium. The reduced proliferation of these cells causes an imbalance between production and loss of epithelial cells, resulting in partial or, in the worst cases, complete vaginal denudation. The early stage can be termed “acute radiation vaginitis”. Edema and inflammation of the epithelium and connective tissues follow and the small blood vessels show endothelial damage and micro-thrombi. The subsequent reduced blood supply, tissue hypoxia, loss of elastin, collagen deposition and hyalinization and fibrosis leads to thinning of the vaginal mucosa, loss of lubrication, scarring and fibrosis. This causes a shorter, less elastic and dryer vagina. The fibrosis results in varying degrees of narrowing, termed vaginal stenosis, and shortening, simply termed “short vagina”. Some women experience complete loss of a functioning vagina. Additionally, estrogen deficiency resulting from radiation-induced menopause or natural menopause, or cessation of prior hormone replacement therapy, may intensify the loss of elasticity and lubrication and thinning and atrophy of the vaginal mucosa (see Miles 2012).

Vaginal radiation injury leading to some degree of vaginal stenosis has been variably estimated to affect between 1.2% and 88% of women subjected to pelvic radiotherapy. Brand et al (2006) found that 38% of women treated for cervical cancer with pelvic and/or vaginal radiotherapy had stenosis (27% partial stenosis or shortening, 11% complete stenosis) at 6-month follow-up. The frequency of stenosis was higher in patients over 50 years old.

Irradiation may take the form of external beam radiotherapy or brachytherapy, applied with an internal vaginal applicator, or both. It may be used alone or after surgery for cervical or endometrial cancer. There is a tendency for vaginal stenosis to occur more frequently with brachytherapy or combined radiotherapy, and when radiotherapy is combined with surgery. The severity of vaginal stenosis appears to be related to a higher dose per fraction of brachytherapy, an increased number of fractions and a smaller diameter of the brachytherapy applicator. It has also been noted that vaginal radiation injury is greater when the entire length of the vagina is irradiated rather than the vaginal apex, and the lower vagina has a poorer tolerance to radiation than the upper vagina.

Current medical treatment of vaginal radiation injury is symptomatic, by means of topical estrogen, topical anti-inflammatory benzydamine and systemic hormones. There is clearly a need for a more effective medical prevention and treatment that is directed at the root cause of the pathology.

### Rectal Radiation Injury (Radiation Proctitis)

The rectum is also subject to radiation injury from pelvic irradiation, which is most commonly given to treat cancers of the uterine cervix, the prostate gland and the rectum. The pathology of the cellular injury is generally similar to that described for the vagina. Free-radical generation leads to apoptosis and disruption of mitosis of the cells of the rectal mucosa, together with fibroblastic proliferation, leading to swelling and sloughing of the mucosa, with ulceration and mucus secretion. This stage is known as “acute radiation proctitis” and clinical symptoms of diarrhea, urgency of defecation and rectal bleeding may appear 2-3 weeks after the start of irradiation. This may settle within a few months or progress to “chronic radiation proctitis”, which may last for years. The initial symptoms persist, with persistent rectal bleeding and the complications of rectal fibrosis, stenosis and fistula-formation. In chronic radiation proctitis, the initial radiation damage to the mucosa is followed by fibrosis and vascular ischemia, compensated for by neovascularization and telangiectasia, leading to the persistent bleeding, while the fibrosis exacerbates the ischemia and may lead to bowel necrosis (Mancini & Sonis 2014).

Radiation proctitis affects approximately 75% of patients subject to pelvic radiotherapy. Current treatment options have very variable, but mostly limited, success rates as reported in small, unblinded clinical studies with variable endpoints. The use of the anti-inflammatory agents 5-aminosalicylic acid and glucocorticoids has been adopted from the treatment of inflammatory bowel disease. Sucralfate has been adopted from its use in the treatment of duodenal ulceration. Metronidazole may be used to mitigate bacterial colonization. Sodium butyrate enemas have been tried, in order to provide a known nutrient factor for cells of the rectal mucosa. None of these therapeutic efforts has proved effective enough to be generally recommended (Denton et al 2002). The mainstay of current treatment of chronic radiation proctitis is endoscopic argon plasma coagulation to control bleeding or radiofrequency ablation for the same purpose. As in the case of radiation vaginitis, there is a need for more effective medical prevention and treatment that is directed at the root cause of the pathology.

### SUMMARY OF THE INVENTION

The invention consists of providing pharmaceutical compositions comprising melatonin or an antioxidant metabolite, derivative or analogue thereof (individually referred to as the protective agent) for the prevention and treatment of vaginal and rectal radiation injury by the direct administration of the compositions to the vaginal and/or rectal epithelium in the form of a foam, gel, cream or ointment, or as a medicated pessary or suppository. The advantage of the invention is that the protective agent is delivered at high dose directly to the tissue for which protection from radiation damage is desired, while there is no direct delivery to the tumor that is to be treated by radiotherapy. A further advantage will be that the directly applied melatonin will not be subject to the low bioavailability of melatonin given orally, which is subject to individually variable first-pass metabolism in the liver. The compositions are intended to be administered immediately before each dose of radiotherapy is given and at various other times during and after a course of radiotherapy.

Pharmaceutical compositions are also provided which comprise melatonin or an antioxidant metabolite, derivative or analogue thereof and a pharmaceutically acceptable form of vitamin E and/or coenzyme Q10 and/or alpha-lipoic acid and/or vitamin C.

Accordingly, the pharmaceutical compositions comprise essentially:
A composition comprising melatonin or an antioxidant metabolite, derivative or analogue thereof formulated to be suitable for administration to the vaginal and/or rectal epithelium as a foam, gel, cream or ointment, or as a medicated pessary or suppository, for the prevention and treatment of vaginal and/or rectal radiation injury.
A composition according to that described above, comprising additionally a pharmaceutically acceptable form of vitamin E and/or coenzyme Q10 and/or alpha-lipoic acid vitamin C.

The invention fulfills the medical need for a preventive, pre-emptive and continuing treatment of the root intracellular cause of radiation injury to the vagina and rectum, for which current treatments are symptomatic or directed at post-hoc alleviation of longer-term pathological consequences.

In the following detailed description of the invention, details of the scope of the invention will be given, together with details of the practical performance of the invention.

### DETAILED DESCRIPTION OF THE INVENTION

The present invention provides compositions comprising melatonin or an antioxidant metabolite, derivative or analogue thereof as the principal active substance to be topically applied to the vaginal and/or rectal epithelium for the prevention and treatment of vaginal and or rectal radiation injury. It also provides for compositions for the same purpose, which additionally comprises a pharmaceutically acceptable form or derivative or analogue of one or more of the substances vitamin E, coenzyme Q10, alpha-lipoic acid and vitamin C.

### Active Ingredients

The principal active ingredient of the compositions of the invention is melatonin or an antioxidant metabolite, derivative or analogue thereof.

### Melatonin

Melatonin (N-acetyl-5-methoxytryptamine) is a hormone produced by the pineal gland in human beings and other mammals by enzymatic modification of the amino acid tryptophan. Melatonin is involved in maintaining the circadian rhythm of various biological functions, being secreted in hours of darkness and acting on high-affinity melatonin Gᵢ-coupled transmembrane receptors MT1 and MT2, which are widely distributed in many cells and tissues of the body. At the same time melatonin acts at supraphysiological concentrations as a powerful antioxidant and free radical scavenger for ROS and reactive nitrogen species (Gomez-Moreno et al 2010). Melatonin can also activate cytoprotective antioxidative enzymes such as copper-zinc and manganese superoxide dismutases (CuZnSOD and MnSOD) and glutathione peroxidase (Rodriguez et al 2004). Melatonin also has anti-inflammatory effects to prevent the upregulation or cause the down-regulation of the expression of nuclear factor kappa B (NF-κB) and pro-inflammatory cytokines such as tumor necrosis factor alpha (TNF-α) and interleukin 1 beta (IL-1β).

Melatonin as an agent to protect against radiation injury: Because of melatonin's efficiency as a free radical scavenger, especially of hydroxyl radicals (Tan et al 1993) and ROS, it has been proposed as an agent to protect against radiation injury to cells and tissues. The protective effect of high dose systemic melatonin against the harmful effects of whole-body irradiation has been studied chiefly in rodents. Melatonin has typically been given at intravenous or intraperitoneal doses of 5 mg to 100 mg per kilogram of body weight and protective effects on DNA and nuclear morphology, as well as prolonged survival after lethal doses of irradiation have been observed. Melatonin has been demonstrated to protect against the adverse effects of all relevant wavelengths ionizing radiation from ultraviolet through x-rays to gamma rays. The results of such studies have been reviewed by Vijayalaxmi et al (2004). In human beings, a protective effect of prior oral melatonin dosage on the damage caused by subsequent ex vivo radiation exposure of lymphocytes has been observed (Vijayalaxmi et al 1996). However, controlled clinical trials of the protective effect of oral or systemic melatonin on radiation damage in patients undergoing radiotherapy are lacking.

The above experiments suggest that a major part of the protective effect of melatonin against radiation damage depends on the intracellular presence of melatonin at the time of radiation. This would be consistent with the near instantaneous intracellular production of free radicals as a result of radiation and their initiation of DNA and mitochondrial damage leading to cell death. There has been some concern that pre-treatment with systemically administered melatonin would also diminish the effectiveness of radiotherapy to kill tumor cells. On the other hand, the effects of melatonin to activate cytoprotective enzymes and down-regulate pro-inflammatory cytokines points to a longer term effect that might contribute to protection against radiation damage.

There are various reports on the use of melatonin as an ingredient in topical skin creams for protection again ultraviolet (UV) irradiation (sun-burn), sometimes in combination with known UV-blocking (sun-screen) agents. Products of this type are available as over-the-counter skin creams. One such agent (PraevoSkin by Praevomed GmbH) has undergone preliminary testing for protection against radiation dermatitis of the chest region in a small number of women undergoing radiotherapy for breast cancer (Ben-David et al 2010). The composition of this preparation, said to be an emulsion containing melatonin, has not been made available. A clinical trial (NCT00840515) was registered in 2009, but has not been reported. This preparation and other over-the-counter melatonin-containing skin preparations have not been proposed for, and are not adapted to vaginal or rectal use.

### Melatonin Derivatives, Analogues and Metabolites

Many chemical derivatives of melatonin, including breakdown products and natural metabolites of melatonin, retain the antioxidant and free-radical scavenging properties of the parent molecule. This makes melatonin a more effective antioxidant than other natural antioxidants such as vitamins C and E (cited by Reiter et al 2007). However, these vitamins show synergy with melatonin with respect to antioxidant activity. In non-hepatic tissues, the reaction of melatonin with two hydroxyl radicals yields the metabolite cyclic 3-hydroxymelatonin (C3-OHM), which undergoes further oxidation by two hydroxyl radicals to break the indole ring and form N¹-acetyl-N²-formyl-5-methoxykynuramine (AFMK) (Tan et al 1993; Reiter et al 2007). C3-OHM is therefore also an effective antioxidant and hydroxyl radical scavenger. The reaction of melatonin with the hydroxyl radical precursor, hydrogen peroxide, similarly leads to production of AFMK. AFMK is also a reducing agent, capable of donating electrons to detoxify radical species, and has been shown to preserve the integrity DNA exposed to oxidizing agents. The action of aryl formamidase or catalase on AFMK produces N¹-acetyl-5-methoxykynuramine (AMK), which is an even more effective scavenger of hydroxyl radicals and reactive nitrogen species, protecting proteins from oxidative destruction. In this process, 3-acetamidomethyl-6-methoxycinnolinone (AMMO) or 3-nitro-AMK (AMNK) is formed.

The liver is the principal site of the classically reported metabolic pathway for melatonin. This consists chiefly of 6-hydroxylation by the cytochromes P450 CYP1A1, CYP1A2, and CYP1B1, and the formation of the minor metabolite N-acetylserotonin by CYP2C19. The main product 6-hydroxymelatonin (6-OHM) is then conjugated at the hydroxyl group to form the 6-OHM glucuronide or 6-OHM sulfate. 6-OHM is an effective free radical scavenger in a variety of situations, but is also reported to show pro-oxidant effects in others. Its status as an antioxidant thus remains equivocal (Maharaj et al 2007).

N-acetylserotonin (NAS) is not only the immediate biosynthetic precursor but also a minor metabolite of melatonin. Like 6-OHM, it is conjugated to form the glucuronide or sulfate. Its protective effect against oxidative damage in certain model systems is reportedly 5 to 20 times as strong as that of melatonin (Oxenkrug 2005).

Melatonin can also be chemically modified by introducing chemical groups at one or more of any of its constituent atoms susceptible of such modification or by introducing such groups in de novo synthesis of melatonin analogues or derivatives. Such modifications, which do not alter the fundamental indole structure of melatonin, are herein called derivatives. The fundamental indole structure of melatonin can also be modified by substituting other bicyclic aromatic structures. Such modifications are herein called analogues, which may also have different chemical side groups removed, introduced or modified. Many such analogues and derivatives have been prepared, but most of them have not been tested for their antioxidant or free-radical scavenging properties.

### Natural Antioxidants that May Act in Synergy with Melatonin

A large number of natural antioxidant agents that have been used pharmaceutically may potentially act synergically with melatonin. These may have additive antioxidant effects, but only a few have been demonstrated to act synergically. Vitamins C and E have been cited in this context and there is preliminary clinical evidence that their systemic administration is beneficial in radiation proctitis (Kennedy et al 2001). A related but not identical property, which is less well assessed, is their efficiency as free radical scavengers and in conferring protection against the harmful effects of radiation and cytotoxic medication. Further natural antioxidants that come under consideration as conferring addition protective effect are alpha-lipoic acid and coenzyme Q10 (also known as ubidecarenone). Both are effective as free radical scavengers and their capacity to ameliorate radiation damage has been demonstrated in vitro and in animal models in which the substances have usually been given intraperitoneally or by dietary supplementation.

Antioxidant metabolites of melatonin: Of those described above, N¹-acetyl-N²-formyl-5-methoxykynuramine (AFMK), 6-hydroxymelatonin (6-OHM) and N-acetylserotonin (NAS) can be used in compositions of the invention. Cyclic 3-hydroxymelatonin (C3-OHM) and N¹-acetyl-5-methoxykynuramine (AMK) are unstable and hence unsuitable for use in a pharmaceutical composition.

Antioxidant melatonin derivatives: The chemical structure of melatonin can be represented as in Figure (I), in which sites suitable for chemical modification by the substitution of different chemical groups have been indicated by R₁, R₂, R₃, R₄, R₅ and R₆. These numbers do not correspond to the conventional numbering of positions in the indole ring of melatonin.

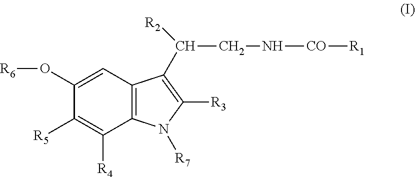

In native melatonin, R₁ and R₆ represent CH₃, while R₂, R₃, R₄, R₅ and R₇ represent H.

Antioxidant melatonin derivatives may comprise, as non-exclusive examples, those in which
R₁ represents H, a linear or branched C₁-C₄ alkyl group or a C₁-C₄ alkoxy group,
R₂ represents H or a C₁-C₄ alkyl group,
R₃ represents H, a methyl group or a halogen atom,
R4 represents H or a halogen atom,
R₅ represents H or a halogen atom,
R₆ represents H or a linear or branched C₁-C₄ alkyl group,
R₇ represents H, a linear or branched C₁-C₄ alkyl group, a —C(═O)-O—Rₐ group or a —C(═O)-N(H)-Rₐ group wherein Rₐ is a linear or branched C₁-C₄ alkyl group, the —CH₂—NH—C(═O)-R₁ side chain is extended by duplicating, triplicating or quadruplicating the —CH₂— group,
   or pharmaceutically acceptable salts of such derivatives.

Synergically acting antioxidants: The present invention also provides compositions comprising melatonin or an antioxidant analogue or metabolite thereof together with a synergically acting antioxidant such as vitamin E, coenzyme Q10, alpha-lipoic acid or vitamin C as active substances. Said substances are herein referred to collectively as the active substances or ingredients. In their native forms, vitamin E is an oil, coenzyme Q10 is an almost water-insoluble solid of low melting point, and alpha-lipoic acid is a water-insoluble solid organic acid, while vitamin C is a solid organic acid. These substances must be provided in forms that are appropriate for the pharmaceutical formulation used. For aqueous carriers, a water-soluble form of vitamin E is D-alpha-tocopheryl succinate. Coenzyme Q10 or a suitable antioxidant analogue or derivative thereof, non-limiting examples of which are coenzyme Q9, decylubiquinone and idebenone, may be rendered water-soluble by adsorption to a biologically acceptable carrier such as beta-cyclodextrin during the formulation process. Alpha lipoic acid R-(+)-alpha-lipoic acid, also called (R)-thioctic acid, can be used as its sodium salt, sodium thioctate, which is soluble in water to yield solutions of near-physiological pH. Similarly, a non-limiting example of an appropriate form of vitamin C is sodium ascorbate.

### Formulations

The pharmaceutical composition of the present invention may be in the form of a foam, gel, cream, ointment, emulsion or suspension, which may be delivered by an appropriate applicator or form part of a medicated pessary or suppository.

The formulation typically contains from 1 mg to 100 mg of melatonin or antioxidant metabolite, derivative or analogue thereof per gram of the composition.

In the preferred embodiment, the formulation is a foam suitable for application to the vagina and rectum. A non-limiting example of such as formulation is a foam based on propylene glycol, comprising additionally the active ingredients, emulsifying wax, polyoxyl 10 stearyl ether and cetyl alcohol, all of pharmaceutical grade. The foam also contains conservation agents such as methyl and/or propyl parahydroxybenzoate, lactic acid or triethanolamine as a pH regulator, purified water and a suitable propellant, a non-limiting example of which is a hydrocarbon propellant such as HP-70, containing isobutane and propane.

A non-limiting example of an oil-based foam is one comprising pharmaceutical-grade mineral oil, the active ingredients and cetyl or cetyl stearyl alcohol, together with a suitable propellant.

Many other compositions of foam preparations suitable for vaginal and rectal use are known to the skilled person and can be applied to the compositions of this invention.

In another embodiment, the formulation is a hydrogel suitable for vaginal and rectal application. A non-limiting example of such as formulation is a hydrogel comprising 20% polyethylene and polypropylene copolymers with Pluronic™ F127 poloxamer and a suitable preservative.

In a further embodiment, the formulation is an ointment suitable for vaginal and rectal application. A non-limiting example of such as formulation is an ointment comprising liquid paraffin, white vaseline, white wax, hydrogenated castor oil and methyl glucose dioleate.

An aspect of these embodiments is that separate formulations may be made for vaginal and rectal use, respectively. One notable difference between the two sites of application is the normal pH level, which is reported to be 3.8-4.5 for the vagina and pH 7-8 for the rectum. Melatonin and the other active ingredients are all stable at these pH ranges. Preparations for vaginal use are accordingly adjusted to have a pH in the range of 3.8-4.5, whereas those for rectal use are adjusted to have a pH in the range of 7-8.

### Administration

Administration of an effective amount of the pharmaceutical composition is by topical application to the vaginal and/rectal epithelium by means of an applicator or insert such as a special pessary or suppository.

In the preferred embodiment, the composition is provided in a spray can that also contains a propellant. When the nozzle of the can is depressed, a valve opens to allow the composition-propellant mixture to escape, so that a foam is formed by the expansion of the propellant on emerging. The spray can nozzle is not applied directly to the vagina or rectum, but to the nozzle of an applicator in the form of a syringe to receive the foam. The applicator nozzle is smooth and of sufficient length to deliver the foam to upper as well lower regions of the vagina or rectum. The foam is applied by hand operation of the applicator syringe to ensure adequate control of the volume and rate of delivery. One gram of composition may expand to about 10 mL of foam.

Gel, cream and ointment preparations are applied with gloved fingers or by means of a smooth applicator. Typically one gram or more, up to 20 grams, of composition may be applied, which has to be dispersed over the whole area of epithelium to be protected.

Compositions which are essentially similar to stiff gels may also be manufactured to coat an appropriately shaped cylindrical pessary or rectal insert made of inert, fibrous material, to place the composition in contact with the epithelium to be protected. The manufacture of such medicated pessaries or inserts are known to those of skill in the art.

### Indications

1. Pelvic external beam radiotherapy for any condition requiring it, including conditions such as cervical carcinoma, endometrial carcinoma, prostatic carcinoma and carcinoma of the rectum or anus.
2. Pelvic brachyradiotherapy for any condition requiring it, including conditions such as cervical carcinoma, endometrial carcinoma, prostatic carcinoma and carcinoma of the rectum or anus.
3. A combination of 1. and 2.

### Dose and Dosage Regimens

By “effective amount” of the pharmaceutical compositions of the present invention is meant a dose, which, when administered to a subject in need thereof, achieves a concentration which has a beneficial biological effect, i.e. by preventing or reducing radiation injury to the vagina and or rectum. Such an effective amount may be determined by physicians of ordinary skill in the art attending patients undergoing pelvic radiotherapy and/or brachytherapy.

The effective amounts and dosages of the ingredients of the composition are not determined in relation to body weight or body surface area, because the treatment is topical to the vagina or rectum.

The effective amount of melatonin or an analogue, derivative or metabolite thereof for a single dose of topical vaginal or rectal administration may be from 1 mg to 100 mg, such as in the range of 1 mg to 50 mg, and especially in the range of 1 mg to 20 mg.

The effective amount of a pharmaceutically acceptable form of vitamin E and/or coenzyme Q10 and/or alpha-lipoic acid and/or vitamin C, in admixture with melatonin or a metabolite, derivative or analogue thereof, may be the same by weight as the amount of melatonin or a metabolite, derivative or analogue thereof.

The effective dose is preferably administered 15 minutes to 30 minutes before each dose of radiation is given. Because melatonin may also have longer term anti-inflammatory effects that are not directly dependent on free radical scavenging, the effective dose may also be given up to six times daily between and after doses of radiation for a period of up to 12 months after the initiation of radiotherapy. The daily dose may be given once a day or in divided or full effective doses two times a day, three times a day, four times a day, five times a day, or six times a day. The total daily dose may thus be from one to six times the amount of a single effective dose.

Duration of dosing will typically range from 3 months to 12 months.

A dose regimen may alternate between periods of administration of the pharmaceutical composition according to the present invention and periods without administration (a pause in treatment). A period with a pause of treatment in such a dose regime may last for 1 week to 2 weeks, or 2 weeks to 3 weeks, or 3 weeks to 1 month, or 1 month to two months, all at the discretion of the attending physician.

### EXAMPLE

The following example illustrates the initial clinical testing of the invention.

### Example 1: Clinical Trial of the Effect of a Composition of the Invention on the Development of Radiation Proctitis

A composition according to the present invention, e.g. a rectal foam containing melatonin, is tested for its efficacy in preventing radiation proctitis by means of a randomized, placebo-controlled, double-blind clinical trial on up to 50 adult patients diagnosed with primary rectal cancer. The patients are all treated with external radiotherapy to reduce tumor size, the total radiation dose of 50.4 Gy being divided into 28 fractions. After giving written, informed consent, the patients are randomized to receive a standard dose of a composition of the present invention or a placebo composition without active ingredients, given rectally 15-30 minutes before each fraction of radiotherapy. The clinician in charge of the trial may also determine that the standard dose should be repeated before retiring each night for the duration of the radiotherapy. The primary outcome is the effect on the Low Anterior Resection Syndrome (LARS) score, a symptom-based scoring system for bowel dysfunction. The LARS score is determined for each patient immediately before starting radiotherapy and again on conclusion of the radiotherapy. All patients have their tumors surgically removed. Secondary outcomes consist of measures of oxidative stress at a cellular level on the excised healthy and tumor tissue. These consist of biochemical markers and pathohistological analysis as determined by the specialized rectal cancer pathologist, who is blinded with respect to the treatment the patient has received.

### REFERENCES

Ben-David M A, Elkayam R, Galarenter I, Pfeffer R M (2010) A prospective, double-blind, randomized study of a melatonin-containing cream for radiation-induced breast dermatitis. ASCO 2010 Breast Cancer Symposium, abstract number 123.

Brand A H, Bull C A, Cakir B (2006) Vaginal stenosis in patients treated with radiotherapy for carcinoma of the cervix. Int J Gynecol Cancer 16:288-293.

Denton A S, Andreyev H J, Forbes A, Maher E J (2002) Systematic review for non-surgical interventions for the management of late radiation proctitis. Br J Cancer 87:134-143.

Gomez-Moreno G, Guardia J, Ferrera M J, Cutando A, Reiter R J (2010) Melatonin in diseases of the oral cavity. Oral Dis 16:242-247.

Kennedy M, Bruninga K, Mutlu E A, Losurdo J, Choudhary S, Keshavarzian A (2001) Successful and sustained treatment of chronic radiation proctitis with antioxidant vitamins E and C. Am J Gastroenterol 96:1080-1084.

Maharaj D S, Glass B D, Daya S (2007) Melatonin: new places in therapy. Biosci Rep 27:299-320.

Mancini M L, Sonis S T (2014) Mechanisms of cellular fibrosis associated with cancer regimen-related toxicities. Front Pharmacol 5:51.

Miles T (ed.) (2012) International guidelines on vaginal dilation after radiotherapy; a clinical guideline. Owen Mumford Ltd, Woodstock, UK.

Oxenkrug G (2005) Antioxidant effects of N-acetylserotonin: possible mechanisms and clinical implications. Ann N Y Acad Sci 1053:334-347.

Reiter R J, Tan D X, Terron M P, Flores L J, Czarnocki Z (2007) Melatonin and its metabolites: new findings regarding their production and their radical scavenging actions. Acta Biochim Pol 54:1-9.

Rodriguez C, Mayo J C, Sainz R M, Antolin I, Herrera F, Martin V, Reiter R J (2004) Regulation of antioxidant enzymes: a significant role for melatonin. J Pineal Res 36:1-9.

Tan D X, Chen L D, Poeggeler B, Manchester L C, Reiter R J (1993) Melatonin: a potent, endogenous hydroxyl radical scavenger. Endocrine J 1:57-60.

Vijayalaxmi, Reiter R J, Herman T S, Meltz M L (1996) Melatonin and radioprotection from genetic damage: in vivo/in vitro studies with human volunteers. Mutat Res 371:221-228.

Vijayalaxmi, Reiter R J, Tan D X, Herman T S, Thomas C R Jr (2004) Melatonin as a radioprotective agent: a review. Int J Radiat Oncol Biol Phys 59:639-653.

1. (canceled)

2. A method for inhibiting a vaginal radiation injury in a subject that has or is at risk of having a vaginal radiation injury due to pelvic radiotherapy, comprising:
selecting a subject that has or is at risk of having a vaginal radiation injury due to pelvic radiotherapy, and
topically administering a formulation comprising melatonin to the vaginal epithelium of the subject;
wherein a single dose of the formulation is topically administered to the subject 15 minutes to 30 minutes before irradiation is initiated.

3. The method of claim 2, wherein the melatonin is formulated as a medicated pessary or suppository.

4. The method of claim 2, wherein the method comprises topically administering the formulation to the vaginal epithelium of the subject 1, 2, 3, 4, 5, or 6 times per day.

5. The method of claim 2, wherein the method comprises topically administering the formulation to the vaginal epithelium of the subject over a period of up to 3 months or more.

6. The method of claim 2, wherein the single standard adult dose of the melatonin is 1 mg to 100 mg.
